(19) United States
(12) Patent Application Publication
(10) Pub. No.: US 2025/0104811 A1
(43) Pub. Date: Mar. 27, 2025

(54) OPTICAL CALIBRATION SYSTEM AND METHOD FOR GENE SEQUENCER

(71) Applicants: NATIONAL INSTITUTE OF METROLOGY, CHINA, Beijing (CN); NORTHEAST ELECTRIC POWER UNIVERSITY, Jilin (CN)

(72) Inventors: Yongzhuo ZHANG, Beijing (CN); Jingjing LIU, Beijing (CN); Jiangyong LI, Beijing (CN); Chunhe WANG, Beijing (CN); Dongfu XU, Beijing (CN); Yifei XU, Beijing (CN); Xingwang LIAN, Beijing (CN); Shixin YU, Beijing (CN)

(21) Appl. No.: 18/888,737

(22) Filed: Sep. 18, 2024

(30) Foreign Application Priority Data
Sep. 27, 2023 (CN) 202311265748.X

(51) Int. Cl. G16B 30/00; G06T 7/80; G09G 3/3208

(52) U.S. Cl. CPC G16B 30/00; G06T 7/80; G09G 3/3208

(57) ABSTRACT

The present invention relates to the field of optical calibration equipment for gene sequencers and discloses a gene sequencer optical calibration system and method, including a control module and a light-emitting calibration module. The light-emitting calibration module is used to simulate the genes measured by a gene sequencer, and the control module is used to control the light-emitting calibration module. The light-emitting calibration module is electrically connected to the control module. The light-emitting calibration module comprises a light-emitting unit and a calibration unit. The light-emitting unit uses multiple OLED screens as the light source. The calibration unit has correction masks for calibration purposes, and the calibration unit is mounted above the light-emitting unit. The present invention solves the problems of complex operations and high costs in the prior art and has the advantages of digital display, flexible switching of calibration structures, no need for calibration solution, and real-time feedback.

### TECHNICAL FIELD

The present invention relates to the field of optical calibration equipment for gene sequencers and more specifically, relates to a gene sequencer optical calibration system and method.

### BACKGROUND OF THE INVENTION

Gene sequencing technology is an important tool for modern biological and medical research. It is of great significance for understanding the nature of life, studying gene functions, diagnosing diseases, etc. However, gene sequencers may experience problems such as optical path deviation, lens focal length abnormality, and imaging distortion after long-time operation or storage. Such optical factors can affect the accuracy and stability of sequencing by instruments, leading to deviations and errors in the sequencing results, and thus jeopardizing the quality and accuracy of the sequencing data.

To improve the accuracy and reliability of gene sequencing technology, researchers need to perform optical calibration on gene sequencers before starting sequencing work formally, to ensure the stability and accuracy of their working state. Optical calibration is a crucial step in gene sequencers, directly affecting the accuracy and reliability of the sequencing results. Traditional optical calibration plates for gene sequencers are typically a combination of biochemical, microfluidic, and micro-nano processing technologies. Flow channels are created inside a calibration plate with microfluidic technology for storing the calibration solution; it is covered with a layer of quartz glass, with two small holes at the bottom of the glass layer serving as passages for injecting or discharging the calibration solution. Masks are deposited on the glass surface in a specific area, and calibration markers and positioning matrices are etched on the masks using micro-nano processing technology. A laser beam is shone through the optical path onto the optical calibration plate, and the calibration solution emits a certain color of light after being excited by the laser light. The microstructure created by micro-nano technology is fed back to the CCD camera of the gene sequencer, and the gene sequencer is calibrated through positioning, recognition, and other operations of the microstructure. This traditional method is complex, requiring advance preparation of optical calibration solutions, and different concentrations of calibration solutions are required for different models of gene sequencers. The applicability of calibration solutions is limited, and calibration plates have to be manually changed between different types, leading to a complex and time-consuming calibration process and making it difficult to achieve flexible calibration.

The prior art includes a mask plate pattern correction method, a mask plate fabrication method, and an optical proximity correction method. The mask plate pattern correction method comprises inspecting the mask plate pattern to determine whether there are areas that will produce side lobes; if not, using the mask plate pattern as the new mask plate pattern with its correction discontinued; if yes, continuing to the next step of correction; adding auxiliary graphics in the areas where the mask plate pattern produces side lobes to form a new mask plate pattern, and re-checking the mask plate pattern until no side lobes appear in the new mask plate pattern.

In summary, the prior art suffers from the problems of complex operations and high costs, so inventing a gene sequencer optical calibration system with simple operations and low costs is an urgent technical need to be addressed in this technical field.

### SUMMARY OF THE INVENTION

The present invention provides a gene sequencer optical calibration system and method to solve the problems of complex operations and high costs in the prior art and has the advantages of digital display, flexible switching of calibration structures, no need for calibration solution, and real-time feedback.

To realize the above purpose of the present invention, the technical solution is as follows:

A gene sequencer optical calibration system comprises a control module and a light-emitting calibration module. The light-emitting calibration module is used to simulate the genes measured by a gene sequencer, and the control module is used to control the light-emitting calibration module. The light-emitting calibration module is electrically connected to the control module. The light-emitting calibration module comprises a light-emitting unit and a calibration unit. The light-emitting unit uses multiple OLED screens as the light source. The calibration unit has correction masks for calibration purposes, and the calibration unit is mounted above the light-emitting unit.

Preferably, it further comprises a human-computer interaction module; the human-computer interaction module is electrically connected to the control module; the human-computer interaction module comprises a display screen and input devices; the human-computer interaction module enters control instructions to the control device via the input devices and shows the system status and control instructions on the display screen.

It further comprises a power supply module; the power supply module is electrically connected to the control module, the light-emitting calibration module, and the human-computer interaction module separately and supplies power to the control module, the light-emitting calibration module, and the human-computer interaction module.

Furthermore, the light-emitting unit specifically comprises two OLED screens; the two OLED screens are respectively set corresponding to the calibration unit.

It further comprises a base; the light-emitting unit is mounted in the base; the base has sockets corresponding to multiple OLED screens; the multiple OLED screens are embedded in the sockets correspondingly and are mounted on the base.

Furthermore, the calibration unit is a quartz glass chip; the correction masks on the quartz glass chip are obtained by depositing metal chromium masks on the quartz glass and then creating the required correction structures on the masks using micro-nano processing technology.

Furthermore, the control device is specifically a Raspberry Pi; the Raspberry Pi is electrically connected to the calibration unit via microHDMI-to-microHDMI signal transmission cables to display the patterns required for optical calibration.

A gene sequencer optical calibration method comprises the following specific steps:
S1: Creating the correction masks of the calibration unit depending on the gene sequencer to be calibrated;
S2: Assembling the calibration unit and the light-emitting unit into a light-emitting calibration module, and setting the pixel resolution, luminous intensity, and spectrum of the multiple OLED screens of the light-emitting unit via the control module;
S3: Controlling the light-emitting unit to emit light via the control module; capturing the correction masks of the calibration unit using the CCD camera of the gene sequencer;
S4: Measuring the offset between the length obtained in the sequencing image of the gene sequencer and the physical length, and calibrating the gene sequencer based on the offset.

Preferably, in step S1, the correction masks of the calibration unit are obtained by depositing metal chromium masks on the calibration unit and then creating the required correction structures on the masks using micro-nano processing technology; the correction masks comprise dark field masks on the perimeter and arranged masks at the center; the dark field masks on the perimeter of the calibration unit are used for horizontal correction; the arranged masks at the center of the calibration unit include masks with micro-nano structures; a mask with a micro-nano structure has a focus crisscross and a square hole matrix; the focus crisscross provides a reference coordinate origin, used to determine the distance, position, and offset parameters between the small holes; the square hole matrix is used to correct the perspective rotation, offset, and imaging distortion of the gene sequencer by measuring the distance between the square holes.

Furthermore, in step S4, the calibration module is run via the control module to control the light-emitting unit to emit light; the specific steps comprising the control module generating a calibration pattern and transmitting it to the pixel points of multiple OLED screens of the light-emitting calibration module, to simulate the state of the base sequence during a real test with the gene sequencer, so as to allow calibration.

The present invention is associated with the following benefits:

The present invention discloses a gene sequencer optical calibration system, comprising a control module and a light-emitting calibration module. The light-emitting calibration module is used to simulate the genes measured by a gene sequencer, and the control module is used to control the light-emitting calibration module. The light-emitting calibration module is electrically connected to the control module. The light-emitting calibration module comprises a light-emitting unit and a calibration unit. The light-emitting unit uses multiple OLED screens as the light source. The calibration unit has correction masks for calibration purposes, and the calibration unit is mounted above the light-emitting unit. The present invention combines the design of OLED screens and correction masks to solve the problems of complex operations and high costs in the prior art, and has the advantages of digital display, flexible switching of calibration structures, no need for calibration solution, and real-time feedback, solving the limitations of traditional calibration plates.

### BRIEF DESCRIPTION OF THE DRAWINGS

FIG. 1 illustrates the modules of a gene sequencer optical calibration system according to the present invention.

FIG. 2 illustrates the control module and OLED screens of a gene sequencer optical calibration system according to the present invention in Embodiment 2.

FIG. 3 illustrates the initial interface of the display screen of a gene sequencer optical calibration system according to the present invention in Embodiment 2.

FIG. 4 illustrates the pixel point sequence state of an OLED screen captured by an optical microscope with a lens magnification of 100 in Embodiment 2.

FIG. 5 illustrates the base of a gene sequencer optical calibration system according to the present invention in Embodiment 2.

FIG. 6 illustrates the correction masks of a gene sequencer optical calibration system according to the present invention in Embodiment 2.

FIG. 7 is a magnified local view of area A of the schematic diagram of the correction masks of a gene sequencer optical calibration system according to the present invention in Embodiment 2.

FIG. 8 is a magnified local view of area B of the schematic diagram of the correction masks of a gene sequencer optical calibration system according to the present invention in Embodiment 2.

FIG. 9 is a magnified local view of area C of the schematic diagram of the correction masks of a gene sequencer optical calibration system according to the present invention in Embodiment 2.

FIG. 10 illustrates the physical installation of the base and the calibration unit of a gene sequencer optical calibration system according to the present invention in Embodiment 2.

FIG. 11 illustrates the flow of a gene sequencer optical calibration method according to the present invention.

In the figures, 1 is the calibration unit, 2 is the base, 3 is the OLED screen socket, and 4 is the FPC cable outlet.

### DETAILED DESCRIPTION OF THE EMBODIMENTS

The following detailed description of the present invention is made with reference to the drawings in specific embodiments.

### Embodiment 1

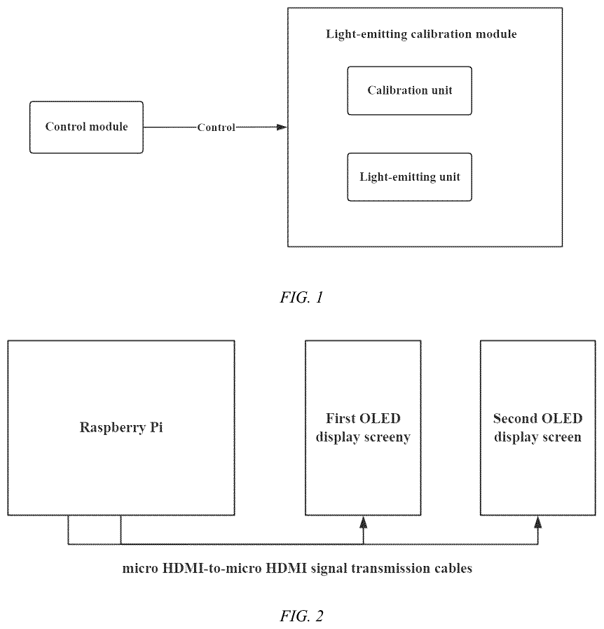

As illustrated in FIG. 1, a gene sequencer optical calibration system comprises a control module and a light-emitting calibration module. The light-emitting calibration module is used to simulate the genes measured by a gene sequencer, and the control module is used to control the light-emitting calibration module. The light-emitting calibration module is electrically connected to the control module. The light-emitting calibration module comprises a light-emitting unit and a calibration unit. The light-emitting unit uses multiple OLED screens as the light source. The calibration unit has correction masks for calibration purposes, and the calibration unit is mounted above the light-emitting unit.

### Embodiment 2

More specifically, in a specific embodiment, it further comprises a human-computer interaction module; the human-computer interaction module is electrically connected to the control module; the human-computer interaction module comprises a display screen and input devices; the human-computer interaction module enters control instructions to the control device via the input devices and shows the system status and control instructions on the display screen.

In this embodiment, the human-computer interaction module provides users with feedback on the display status of the base sequence image simulated by the calibration unit 1 and commands to indicate input image changes;

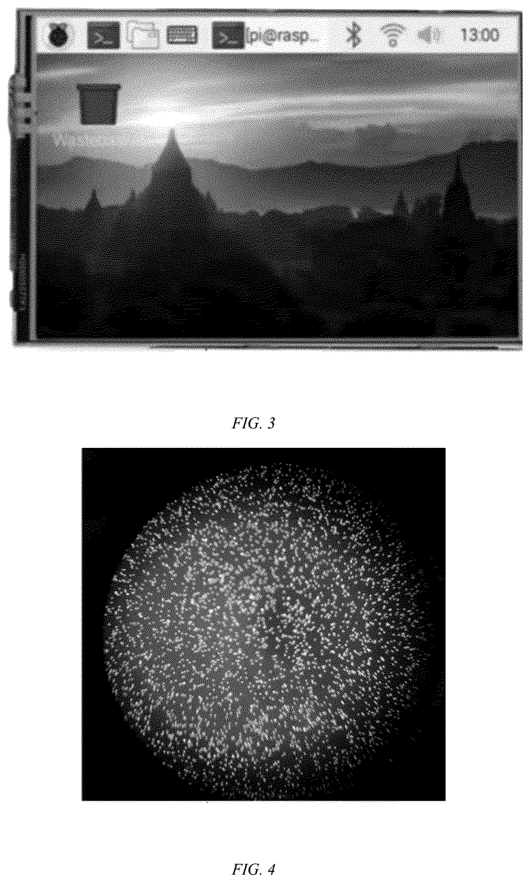

Specifically, as illustrated in FIG. 2, the control device is specifically a Raspberry Pi; the Raspberry Pi is electrically connected to the calibration unit 1 via microHDMI-to-microHDMI signal transmission cables to display the patterns required for optical calibration. In this embodiment, the light-emitting unit comprises a first OLED display screen and a second OLED display screen; during operation, the input devices of the Raspberry Pi comprise peripherals such as a keyboard and a mouse and realize instruction input, image display, and other operations for the calibration device. The display screen is a touch screen; the touch screen provides system operation information to the user through the on/off status of indicator lights of different colors; the touch screen displays and feeds back the execution status of the control of the OLED screens in real time; the initial interface of the touch screen is illustrated in FIG. 3; the image displayed on an OLED screen is illustrated in FIG. 4.

In a specific embodiment, it further comprises a power supply module; the power supply module is electrically connected to the control module, the light-emitting calibration module, and the human-computer interaction module separately and supplies power to the control module, the light-emitting calibration module, and the human-computer interaction module. In this embodiment, the power supply module uses a 15W or higher power supply to power the system equipment with the green light indicating that the power supply works normally, and the power supply should have good load stability and anti-interference performance.

In a specific embodiment, as illustrated in FIG. 3, the light-emitting unit specifically comprises two OLED screens; the two OLED screens are respectively set corresponding to the calibration unit 1, and the OLED screens are embedded in the sockets corresponding to the calibration unit.

In this embodiment, the control system uses Raspberry Pi 4B as the PC end, which has a processor with a main frequency of 1.5 Ghz, a memory of 4G, a dual-band 2.4G/5G Wi-Fi module, Bluetooth 5.0, and support for 4K dual displays. It has four USB ports for connecting peripherals, such as a mouse and a keyboard. It has two microHDMI video output ports, which can output signals to two displays. It is connected to the OLED screens' driver board via microHDMI-to-microHDMI signal transmission cables to display the specific patterns required for optical calibration. After the Raspberry Pi 4B is powered on, the indicator lights will light up; the red light is the power indicator, and the green light is the system operation indicator and will blink irregularly when booting up. It flashes once occasionally after the bootup is complete. After powering on, the Raspberry Pi 4B starts, the 3.5-inch touchscreen display lights up with an image, and after the booting process is completed, it will enter the system interface. The OLED screen is a 0.39-inch high-brightness screen with a resolution of 1024*768 pixels and a maximum brightness of 1,000 cd/m². It is driven by an active matrix color OLED chip based on monocrystalline transistors, with a precise active drive mode that drives and controls the light-emitting array with good optical consistency and fluorescence simulation. By running a calibration program, various sequences and images with known parameters, such as light intensity, are generated and displayed on the two OLED display screens. OLED screens can effectively simulate the light-emitting state of bases during gene sequencing and can also be used as backlight panels for optical calibration.

In this embodiment, the two OLED screens establish a three-dimensional spatial layout with a good optical effect with the calibration unit 1.

In the layout, to produce a good optical effect: the premise of using OLED screen self-emission instead of emission of traditional calibration solutions by laser irradiation is that the relevant optical indicators (for example, light intensity and spectrum) of OLED screen emission should be consistent with the optical indicators of emission of traditional calibration solutions by laser irradiation for it to have practical significance for calibration of optical indicators of gene sequencers. According to literature queries, the optical indicators during real sequencing include light intensity of 20-40 mcd, red primary wavelength of 590-625, and green primary wavelength of 530-560 nm. Therefore, the optical indicators of the OLED screens should also be consistent with these. Since the light intensity decays as the distance increases, the distance between the OLED screens and the calibration unit 1 is calculated using a formula so that the light loss rate is within an acceptable range. The spectrum itself does not decay, but light may be absorbed, scattered, and attenuated during propagation and passage through various media, so quartz glass with good optical performance is chosen for the calibration unit 1. After the processing and arrangement are completed, specialized detection equipment is used to monitor the optical indicators. The results show that after the light propagates, all the indicators still meet the requirements when the OLED screens work with the calibration unit 1. That is, the OLED screens and the optical calibration unit 1 produce a good optical effect.

A three-dimensional spatial layout with a good optical effect: The two OLED screen light-emitting units and the calibration unit 1 should maintain a good layout in three-dimensional space so that they maintain the optimal horizontal state and a good corresponding state between the specific masks and the OLED screens. The calibration unit 1 is arranged with a number of masks, and micro-nano structures are created on the masks. In the layout, it is necessary to ensure that at least one mask of each structure type corresponds to the OLED screen. For different models of sequencers, the layout can be arranged according to this principle. The structure cannot be adjusted once finalized.

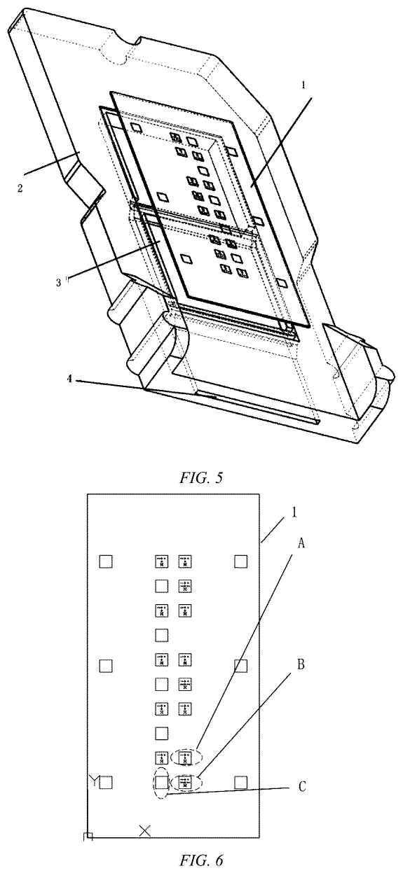

In a specific embodiment, as illustrated in FIG. 5, it further comprises a base 2; the light-emitting unit is mounted in the base 2; the base 2 has sockets 3 corresponding to multiple OLED screens; the multiple OLED screens are embedded in the sockets 3 correspondingly and are mounted on the base 2.

In this embodiment, the two OLED screens mounted in the base 2 are electrically connected to the Raspberry Pi through the FPC cable outlet 4.

In this embodiment, considering the spatial layout of the OLED screens and the optical calibration chip, as well as the required optical performance, the base drawings of the OLED screens and quartz glass are designed. The base is designed considering factors such as light signal attenuation and interference, as well as the microstructure of the quartz glass. Experiments have proven that this structure has good optical consistency and optical performance. The relative position of the OLED screens is adjusted according to the relative position of the masks etched on the optical calibration chip so that the masks are illuminated as much as possible by the backlight source of the OLED screens.

In a specific embodiment, the calibration unit 1 is a quartz glass chip; the correction masks on the quartz glass chip are obtained by depositing metal chromium masks on the quartz glass and then creating the required correction structures on the masks using micro-nano processing technology.

In this embodiment, the required quartz glass thickness is determined according to the selected optical performance indicators of the OLED screens, usually being 100 um-500 um. As illustrated in FIG. 6, the thickness of the quartz glass chip used in this embodiment is designed to be 170 um. Mask metal chromium is deposited on a specific location of the quartz glass, and the mask thickness is usually 50 nm-750 nm. The mask thickness used in this embodiment is 200 nm. Then, interference patterns such as calibration markers and square hole matrices are created with technologies such as Steeper lithography and IBE; the quartz glass is prepared with high-precision processes and equipment to meet the requirements for alignment accuracy, dimensional accuracy, and optical performance. In gene sequencers, the surface flatness of the calibration plate should meet a certain level to ensure the accuracy and stability of the optical path.

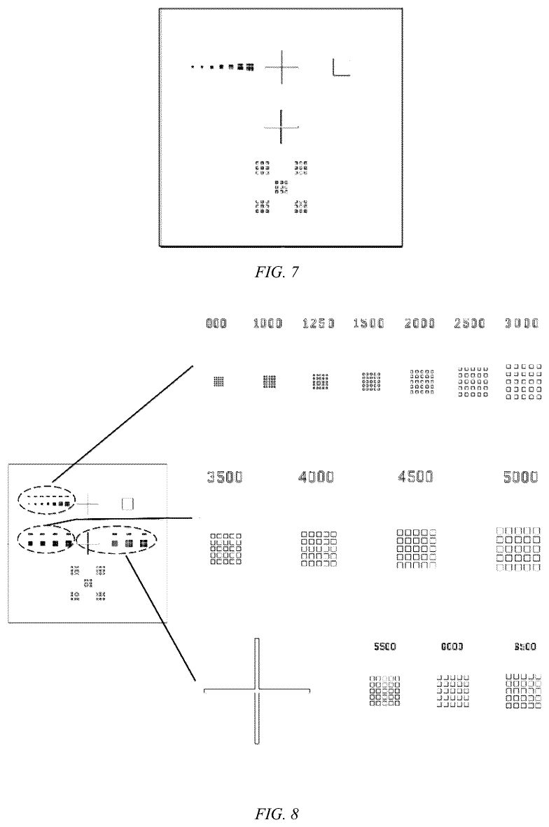

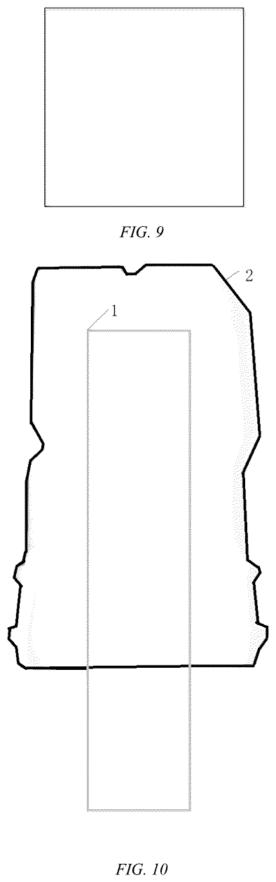

In this embodiment, as illustrated in FIGS. 7, 8, and 9, the quartz glass surface is etched with a mask metal pattern, which is a transparent or semi-transparent square mask. Some black or dark gray squares or straight lines are etched on the surface to calibrate the CCD camera, optical path, or laser system of the gene sequencer to ensure the quality and accuracy of images. The etched patterns mainly include calibration markers, square hole matrices, and dark field masks. The calibration marker pattern provides a reference coordinate system when the CCD camera captures images of the calibration plate. The reference coordinate system can be used to determine parameters such as the distance, position, and offset between the small holes and can also be used to correct problems such as perspective rotation, offset, and deformation that occur during sequencing. Due to the accuracy of the lens in manufacturing and the effect of other components along the optical path, the actual focal length may deviate slightly from the theoretical value and can be corrected by measuring the distance between the small holes using the calibration plate. The lens, other components in the optical path, and imaging chips may cause imaging distortion, meaning that the actual length in the image is not consistent with the physical length. The optical calibration plate can correct distortion by measuring the offset of the position of small holes. Background noise may affect the fluorescence signal intensity during sequencing. Calibration using a dark field mask pattern can eliminate the effect of background noise on signals, thereby improving the precision and accuracy of signal recognition.

In this embodiment, the etched patterns include calibration crisscrosses, square hole matrices, and dark field masks, with a minimum size of 800 nm and a maximum size of 6,500 nm. They are combined with distributed OLED screens to optically calibrate most gene sequencers available on the market. The numbers engraved above the square hole matrices etched on the masks of the calibration unit 1 indicate the geometric dimensions of the square hole matrices, being 800 nm, 1,000 nm, 1,250 nm, 1,500 nm, 2,000 nm, 2,500 nm, 3,000 nm, 3,500 nm, 4,000 nm, 4,500 nm, 5,000 nm, 5,500 nm, 6,000 nm, and 6,500 nm.

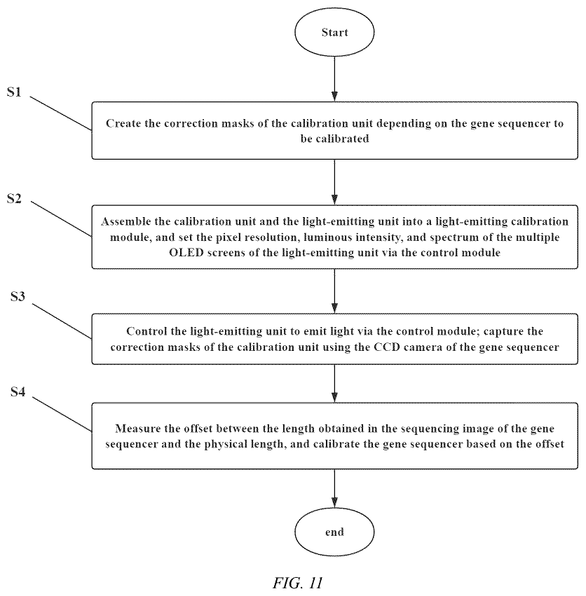

As illustrated in FIG. 10, the method proposed in the present invention has the following advantages and effects:

Traditional optical calibration plates have flow channels inside, requiring the injection of prepared calibration solutions, and then optical parameter calibration is performed by laser irradiation combined with the microstructure on the surface glass chip. The present invention proposes using OLED screens as backlight sources and metal etched patterns on masks as correction structures and calibrating relevant parameters of the CCD camera, optical paths, and light sources through calibration markers, square hole matrices, and dark field masks.

The developed optical calibration equipment based on OLED screens can achieve calibration of light sources, CCD cameras, and optical paths by selecting specific pixel images based on calibration procedures or indicators and displaying them on OLED screens as backlight sources, combined with mask microstructures etched on special quartz glass.

The relevant optical indicators of OLED screens are substantially consistent with those of bases during gene sequencing, which can effectively simulate the sequencing state, thereby achieving optical indicator calibration without calibration solution, simplifying the calibration process. Multiple calibration images and square hole matrices in different sizes etched on the masks meet the needs of optical calibration for various types of gene sequencers.

For quartz glass calibration unit 1, micro-nano processing technology is used to deposit mask metal chromium on quartz glass with a specific thickness and then process microstructures on the masks.

Optical calibration is critical in the sequencing process. With the calibration plate in place, the system can accurately locate the optical path and calibrate the errors between different detection systems (such as cameras and laser sources), ensuring the accuracy and reliability of the sequencing data.

### Embodiment 3

As illustrated in FIG. 11, a gene sequencer optical calibration method comprises the following specific steps:
S1: Creating the correction masks of the calibration unit depending on the gene sequencer to be calibrated;
S2: Assembling the calibration unit and the light-emitting unit into a light-emitting calibration module and setting the pixel resolution, luminous intensity, and spectrum of the multiple OLED screens of the light-emitting unit via the control module;
S3: Controlling the light-emitting unit to emit light via the control module; capturing the correction masks of the calibration unit using the CCD camera of the gene sequencer;
S4: Measuring the offset between the length obtained in the sequencing image of the gene sequencer and the physical length, and calibrating the gene sequencer based on the offset.

In a specific embodiment, in step S1, the correction masks of the calibration unit are obtained by depositing metal chromium masks on the calibration unit and then creating the required correction structures on the masks using micro-nano processing technology; the correction masks comprise dark field masks on the perimeter and arranged masks at the center; the dark field masks on the perimeter of the calibration unit are used for horizontal correction; the arranged masks at the center of the calibration unit include masks with micro-nano structures; a mask with a micro-nano structure has a focus crisscross and a square hole matrix; the focus crisscross provides a reference coordinate origin, used to determine the distance, position, and offset parameters between the small holes; the square hole matrix is used to correct the perspective rotation, offset, and imaging distortion of the gene sequencer by measuring the distance between the square holes.

In this embodiment, the focus crisscross can be used to adjust the focal length of the CCD camera. The perspective rotation, offset, and imaging distortion of the gene sequencer can be corrected by measuring the distance between the square hole matrices.

In a specific embodiment, in step S4, the calibration module is run via the control module to control the light-emitting unit to emit light; the specific steps comprising the control module generating a calibration pattern and transmitting it to the pixel points of multiple OLED screens of the light-emitting calibration module for them to display red or green or be not lit, to simulate the state of the base sequence during a real test with the gene sequencer, so as to allow calibration.

In this embodiment, measuring the offset between the length obtained in the sequencing image of the gene sequencer and the physical length and calibrating the gene sequencer based on the offset are done in the following steps: The CCD camera of the gene sequencer is used to capture the calibration unit; the geometric parameters in the captured image are measured; if there is an offset from any geometric parameter of the calibration unit, adjustments are made until they are consistent. Alternatively, a Raspberry Pi can be used to generate pixel points in a known sequence, which are then captured by the CCD camera, and the pixel point sequence is decoded and then compared with the known generated pixel point sequence to figure out the sequencing accuracy. Calibration is performed if the accuracy is low. Background noise may affect the light signal intensity during sequencing. Calibration using dark field masks can eliminate the effect of background noise on signals, thereby improving the precision and accuracy of signal recognition. Overall, the calibration structures include horizontal correction masks, dark field masks, focus crisscrosses, square hole matrices of different sizes, and pixel point sequences. Optical calibration using these structures substantially solves the optical problems encountered by gene sequencers.

In this embodiment, a Raspberry Pi is used as the main controller to generate a calibration pattern, which is then transmitted to the OLED screens. Each pixel point of the OLED screens is controlled to display red or green or be not lit to simulate the state of the base sequence during a real test with the gene sequencer so as to allow calibration. The function of the square hole matrices is not only to correct problems such as perspective rotation, offset, and imaging distortion of the gene sequencer. It can also be combined with the pixel points of the OLED screens to form various known sequences for detection by the gene sequencer. The pixel size of the OLED screens is 5.5 um. To generate a known sequence of 800 nm, pixel points are lit according to the interval, and then the light passes through the 800 nm square hole matrix, thereby generating a pixel point sequence of 800 nm. Other sequences can be generated likewise.

It is evident that the embodiments of the present invention described above are merely illustrative examples for the purpose of clarifying the present invention and are not intended to limit the scope of embodiments of the present invention. Any modifications, equivalent replacements, and improvements made without departing from the spirit and scope of the present invention shall be included in the protected scope of the claims of the present invention.

1. A gene sequencer optical calibration system, characterized in that, it comprises a control module and a light-emitting calibration module; the light-emitting calibration module is used to simulate the genes measured by a gene sequencer, and the control module is used to control the light-emitting calibration module; the light-emitting calibration module is electrically connected to the control module; the light-emitting calibration module comprises a light-emitting unit and a calibration unit; the light-emitting unit uses multiple OLED screens as the light source; the calibration unit has correction masks for calibration purposes, and the calibration unit is mounted above the light-emitting unit.

2. A gene sequencer optical calibration system according to claim 1, characterized in that, it further comprises a human-computer interaction module; the human-computer interaction module is electrically connected to the control module; the human-computer interaction module comprises a display screen and input devices; the human-computer interaction module enters control instructions to the control device via the input devices and shows the system status and control instructions on the display screen.

3. A gene sequencer optical calibration system according to claim 2, characterized in that, it further comprises a power supply module; the power supply module is electrically connected to the control module, the light-emitting calibration module, and the human-computer interaction module separately and supplies power to the control module, the light-emitting calibration module, and the human-computer interaction module.

4. A gene sequencer optical calibration system according to claim 1, characterized in that, the light-emitting unit specifically comprises two OLED screens; the two OLED screens are respectively set corresponding to the calibration unit.

5. A gene sequencer optical calibration system according to claim 1, characterized in that, it further comprises a base; the light-emitting unit is mounted in the base; the base has sockets corresponding to multiple OLED screens; the multiple OLED screens are embedded in the sockets correspondingly and are mounted on the base.

6. A gene sequencer optical calibration system according to claim 1, characterized in that, the calibration unit is a quartz glass chip; the correction masks on the quartz glass chip are obtained by depositing metal chromium masks on the quartz glass and then creating the required correction structures on the masks using micro-nano processing technology.

7. A gene sequencer optical calibration system according to claim 1, characterized in that, the control device is specifically a Raspberry Pi; the Raspberry Pi is electrically connected to the calibration unit via a microHDMI-to-microHDMI signal transmission cables to display the patterns required for optical calibration.

8. A gene sequencer optical calibration method, characterized in that, it comprises the following specific steps:
S1: Creating the correction masks of the calibration unit depending on the gene sequencer to be calibrated;
S2: Assembling the calibration unit and the light-emitting unit into a light-emitting calibration module and setting the pixel resolution, luminous intensity, and spectrum of the multiple OLED screens of the light-emitting unit via the control module;
S3: Controlling the light-emitting unit to emit light via the control module; capturing the correction masks of the calibration unit using the CCD camera of the gene sequencer;
S4: Measuring the offset between the length obtained in the sequencing image of the gene sequencer and the physical length, and calibrating the gene sequencer based on the offset.

9. A gene sequencer optical calibration method according to claim 8, characterized in that, in step S1, the correction masks of the calibration unit are obtained by depositing metal chromium masks on the calibration unit, and then creating the required correction structures on the masks using micro-nano processing technology; the correction masks comprise dark field masks on the perimeter and arranged masks at the center; the dark field masks on the perimeter of the calibration unit are used for horizontal correction; the arranged masks at the center of the calibration unit include masks with micro-nano structures; a mask with a micro-nano structure has a focus crisscross and a square hole matrix; the focus crisscross provides a reference coordinate origin, used to determine the distance, position, and offset parameters between the small holes; the square hole matrix is used to correct the perspective rotation, offset, and imaging distortion of the gene sequencer by measuring the distance between the square holes.

10. A gene sequencer optical calibration method according to claim 8, characterized in that, in step S4, the calibration module is run via the control module to control the light-emitting unit to emit light; the specific steps comprising the control module generating a calibration pattern and transmitting it to the pixel points of multiple OLED screens of the light-emitting calibration module, to simulate the state of the base sequence during a real test with the gene sequencer, so as to allow calibration.
